(19) United States
(12) Patent Application Publication
(10) Pub. No.: US 2014/0135819 A1
(43) Pub. Date: May 15, 2014

(54) TOURNIQUET AND METHOD OF USE

(71) Applicant: Alice Brown, Saginaw, MI (US)

(72) Inventor: Alice Brown, Saginaw, MI (US)

(21) Appl. No.: 13/838,477

(22) Filed: Mar. 15, 2013

Related U.S. Application Data
(60) Provisional application No. 61/724,805, filed on Nov. 9, 2012.

(51) Int. Cl. A61B 17/132

(52) U.S. Cl. CPC A61B 17/1325
USPC 606/203

(57) ABSTRACT

A tourniquet includes a flexible and stretchable strap of neoprene material supporting a relatively rigid stay on an inner surface of the strap. A buckle is secured to one end of the strap and the other end is looped through the buckle and can be tightened about a person's limb at a location of a severe wound where there is severe external or internal bleeding that must be stopped. The stay is position at or above the bleed site and the strap tightened enough to apply sufficient cuff pressure by the stay to stop the bleeding, while the flexible and stretchable strap applies a relatively lower cuff pressure to permit continued venous blood flow across the strap in the surrounding uninjured region of the limb.

This application claims priority to U.S. application Ser. No. 61/724805, filed Nov. 9, 2012, and is incorporated herein by reference.

### BACKGROUND OF THE INVENTION

1. Technical Field

This invention relates generally to tourniquets and to methods of using such devices.

2. Related Art

Tourniquets are sometimes used in emergency and/or combat situations as a device to stop severe bleeding of limb of a badly injured person, such as a combat soldier or someone who has suffered a gunshot wound, a severe laceration or internal bleeding. For military applications, the Combat Application Tourniquet (a.k.a. C-A-T) made by Composite Resources, Inc. is often utilized, and this device employs a nylon strap arrangement in combination with a windlass and locking mechanism that act to apply a tight, uniform constricting force about the limb at pressures (cuff pressure or limb occlusion pressure) of about 400 mm/Hg. This cuff pressure is well above the 300 mm/Hg minimum required to stop arterial blood flow. There are other improvised tourniquets that are sometimes used and which function in similar manner (albeit it less sophisticated than the C-A-T device), wherein a belt, shirt, shoe lace, handkerchief or similar device is wrapped about the circumference of the injured limb at a location above the wound and is tightened, often with the aid of a stick, pipe or other rigid windlass device, to exert sufficient, uniform cuff pressure on the limb to stop the severe bleeding , including arterial bleed.

While such devices, when properly used, can be very effective in stopping blood flow of an injured extremity and keeping a severely wounded person from bleeding to death, the use of such tourniquets is considered by some to be a life-over-limb choice, since their use is often associated with the loss of the affected limb. The perception is that the total stoppage of blood flow to the limb at the location below the tourniquet can cause irreversible damage to the tissues of the affected limb due to the build-up of toxins in the blood that is trapped in the limb by the tourniquet, and while it may save a person's life, it may be at the expense of the limb. There are also nerves than are bundled close to arteries and through the limb, and the high compression force of traditional tourniquets can interfere with their function and in some cases can be damaged, contributing to the trauma to the leg.

In addition to arterial blood flow to the limbs, the human circulatory system also includes venous blood flow, which returns oxygen depleted blood from the tissues of the limbs back to the lungs. The cuff pressure needed to stop venous blood flow is only about 200 mm/Hg, and thus usage of the traditional tourniquets described above, which apply uniform constricting cuff pressures about the full circumference of the limb in excess of 300 mm/Hg in order to stop arterial bleed, also act to completely block off venous blood flow to the limb even though the veins themselves may not have been bleeding. It is also recognized that an artery extending into a limb (such as the femoral artery of a leg) braches off from the main artery into secondary arteries and those in turn branch off. It is not unusual for one or more of the branch arterial to be partially or completely severed, while the remaining branches may be intact and functioning at the time of the injury. However, in addition to completely blocking venous flow, the application of traditional tourniquets described above also may partially or entirely block arterial flow of the undamaged branches of the arteries since they may be closed off by the high uniform constricting force applied by the traditional-type tourniquet. As such, traditional tourniquets impair or completely block collateral arterial blood flow through the undamaged arteries.

This complete blockage of venous blood flow and/or collateral arterial blood flow and/or severe compression of the nerves of the limb to which a traditional tourniquet is applied can cause severe discomfort to the victim. The prolonged starvation of a limb of arterial and/or venous blood flow and/or severe or prolonged compression of the nerves by the traditional tourniquet may cause irreversible damage to the affected tissues and may lead to extensive tissue debridement and/or loss of the affected limb. As such, it is the perception of many that the tradeoff of using a tourniquet as a life-saving measure to stop arterial bleed of a limb may be the loss of the limb. And considering that there may be a relatively short window of time available for a traumatically injured person to decide whether or not to apply a tourniquet to an injured limb suffering severe arterial bleeding (on the order of about 30 seconds), such person may choose not to use the tourniquet for fear of losing a limb or by the time they do decide it may be too late to save them from bleeding to death.

### SUMMARY OF THE INVENTION AND ADVANTAGES

A tourniquet device is constructed and effective to apply dual pressure to an injured limb, such that a localized higher cuff pressure is applied to a wound site to stop localized severe bleeding, while a reduced cuff pressure is applied to the surrounding uninjured regions of the limb to maintain blood flow away from the wound site.

The dual pressure device addresses the perceived “life-or-limb” decision one may face when deciding whether or not to place a tourniquet on a severely injured limb. The device offers the life-saving benefits of using a tourniquet to stop arterial bleed, while reducing and the possible amputation of the limb caused by irreversible damage to the tissues of the limb from lack of sufficient blood flow resulting from use of a tourniquet. The device according to one aspect of the invention is designed to apply sufficiently high pressure to the arterial or other region of the limb suffering from severe bleeding in order to achieve occlusion of the affected area to stop the bleed, while applying considerably less pressure to the remainder of the limb to enable continued venous flow and possibly continued partial (collateral) arterial blood flow across the tourniquet which is effective to preserve the tissue and significantly decrease the chances for loss of limb.

The invention recognizes that venous flow in a limb will continue even in the presence of arterial bleed, so long as the cuff pressure (or limb occlusion pressure) of an applied tourniquet is less than 250 mm/Hg, keeping in mind that the minimum cuff pressure to stop arterial bleed is 300 mm/Hg. While standard tourniquets apply a uniform circumferential cuff pressure of 300 mm/Hg or more to achieve the stoppage of arterial bleed, it is at the expense of cutting off venous flow and can lead to undesirable dame or loss of limbs. This is not so with the present tourniquet.

An exemplary tourniquet device includes a first portion that is capable of applying no more than 250 mm/Hg cuff pressure to the tissues of the limb. The exemplary tourniquet device further includes a second portion that is coupled to the first portion and is effective to apply localized cuff pressure in the contact region of the second portion of more than 300 mm/Hg. Such a device is able to stop arterial bleed in the affected area while maintaining venous flow and maintaining arterial flow if present in unaffected regions of the limb.

An exemplary tourniquet device is constructed to apply variable cuff pressure about the circumference of a limb, with a sufficiently high pressure being applied in the region of the severe bleed site to stop the bleeding, and with relatively lower cuff pressure being applied away from the artery sufficient to maintain blood flow to the unaffected regions of the limb across the lower cuff pressure portion of the tourniquet in order to preserve the healthy tissue of the limb.

An exemplary tourniquet device includes a first strap portion that is flexible and elastic and effective to apply 250 mm/Hg or less cuff pressure to a limb to which it is applied, and a second relatively rigid portion coupled to the first portion and effective to apply 300 mm/Hg or more cuff pressure to a region of the limb that is acted upon by the second portion. The rigid portion has a smaller contact surface area with the limb than that of the stretchable elastic strap portion

According to a further aspect, the second portion of such a device is effective to impart cuff pressures of up to 600 mm/Hg or more to stop arterial blood flow while the first portion is effective to impart only 150 mm/Hg or less to maintain venous blood flow of the limb.

An exemplary tourniquet device takes into account surface area, material properties, elasticity, inelasticity, stretchability and their individual and combined effects on the cuff pressure exerted on the limb by a wrap. The exemplary tourniquet includes a flexible, stretchable strap portion that is wrappable about at least a portion of the circumference of a person's limb and which applies a cuff pressure of 250 mm/Hg or less, and a relatively rigid, narrow, bar portion supported by the strap portion and due to its rigidity and reduced surface area imparts a significantly higher effective cuff pressure to the limb where it makes contact, such that while the flexible strap applies a relatively low cuff pressure (250 mm/Hg or less) to maintain venous blood flow to preserve the limb tissues and minimize the chance of amputation, the rigid, narrow bar portion exerts a cuff pressure in excess of 300 mm/Hg in order to stop arterial blood flow or other severe bleeding when positioned across the affected arterial or other area at or above above the bleed site.

According to an exemplary embodiment, the flexible, stretchable strap portion is made of neoprene material and the relatively rigid narrow bar portion is made of a cylindrical length of plastic, metal, ceramic, wood, or other suitable material.

The relatively rigid portion material may be hard and may be elastically flexible, malleable and selectively shapeable, or stiff and inelastic.

The relatively rigid portion has a length measurement that is greater than its width measurement. The length may be ten times or more greater than its width. The width may be 1 inch outer diameter or less. The width may be ¼ inch in outer diameter. The length may be 2 inches to 6 inches. The length may be 4 inches.

The relatively rigid portion extends in the lengthwise (circumferential) direction of the flexible stretchable strap for only a relatively small portion of the overall length of the strap. The relatively rigid portion is secured in position on the strap. The relatively rigid portion may be secured in a pocket of the flexible strap. The relatively rigid portion may be selectively detachable from the strap, or permanently attached. It may be captured in a pouch that is permanently or removable secured to the strap (such as sewn on or secured removably by hook and loop fasteners, for example.

The flexible, stretchable strap includes a strap securement system for securing the strap in position about the circumference of a user's limb. The securement system may include hook and loop fastening portions. The securement system may further include a buckle device secured to the flexible stretchable strap and constructed to receive a free end portion of the strap threaded therethrough to help support the strap while being tightened about the limb with one-handed application, if necessary.

In use, the strap is wrapped about a user's limb at a location at or above a partially or fully severed artery or severe bleed region (e.g., gunshot wound or severe laceration). The relatively rigid portion is positioned across the artery or bleed site so as to lie cross-wise to the artery or other bleed site to be blocked. The flexible stretchable strap is tightened about the circumference of the limb to exert a cuff pressure of less than 200 mm/Hg. The relatively narrower and more rigid stay or rigid portion concentrates the cuff pressure in a smaller surface area where it contact the limb to effectively amplify the localized cuff pressure across the artery or other bleed site to above 300 mm/Hg. Such tourniquet is effective in stopping arterial or other severe bleed by virtue of the localized applied arterial cuff pressure of +300 mm/Hg, while maintaining venous blood flow and possibly arterial flow through undamaged arterials of the limb away from the affected region by virtue of the cuff pressure of the flexible and stretchable strap being below 200 mm/Hg and thereby permitting continued flow of blood to and from the limb.

### BRIEF DESCRIPTION OF THE DRAWINGS

FIG. 1 is an perspective view of the tourniquet applied to a person's limb;

FIG. 2 is a top view of the tourniquet;

FIG. 3 is a bottom view of the tourniquet;

FIG. 4 is a cross sectional view through a limb with the tourniquet applied;

FIG. 5 is an enlarged fragmentary cross-sectional view of a portion of the tourniquet;

FIG. 6 is a perspective view of a stay portion of the tourniquet;

FIG. 7A-7E are enlarged cross sections of alternative profiles of the stay of FIG. 6;

FIG. 8 is a perspective view of an alternative tourniquet device; and

FIG. 9 is an enlarged fragmentary cross-sectional view of a portion of the device of FIG. 8.

### DETAILED DESCRIPTION

A tourniquet device 10 includes a strap portion, or strap 12 and a rigid stay portion or rigid member 14. The device 10 is designed to apply a variable, or dual, cuff pressure to a user's injured limb, with the stay portion offering a relatively high cuff pressure to stop blood flow due to an injury, and the balance of the strap portion offering a relatively low cuff pressure sufficient to support the tourniquet in place wrapped about the limb while allowing venous blood circulation to continue in the surrounding uninjured regions of the limb across the low pressure strap portion.

The strap 12 is of sufficient length to enable the strap to be wrapped about the circumference of a person's limb. The strap 12 may have a length of from 8 inches to 36 inches. The exact length of the strap 12 is not critical so long as it fits about the user's injured limb, and it will be appreciated by those of ordinary skill in the art that the desired length can be chosen to fit a particular person or general size of person (e.g., small, medium, large and/or extra large) so as to have a selection of such devices with straps of differing length, or the length can be chosen to fit limbs of a variety of sizes (e.g., adult biceps in the case where the limb is an arm to adult thighs in the case where the limb is a leg, and of various sizes thereof). The effective length of the strap 12 can be adjustable through use of a strap buckle and strap retainer system, as will be explained in greater detail below.

The rigid stay portion 14 is carried on the strap 12 on an inner surface thereof and projects outwardly of the inner surface of the strap 12, and has a predetermined length that is considerably less than that of the strap 12. The length of the rigid stay portion 14 may be on the order of 2 to 6 inches and thus extends over only a small portion of the length of the strap 12. The length may be 4 inches. The stay portion 14 extends parallel to the lengthwise direction of the strap 12. The stay portion 14 may constitute an integral part of the strap (e.g., may be molded or formed in place on the strap) or may be separately made and secured permanently or removably to the strap 12.

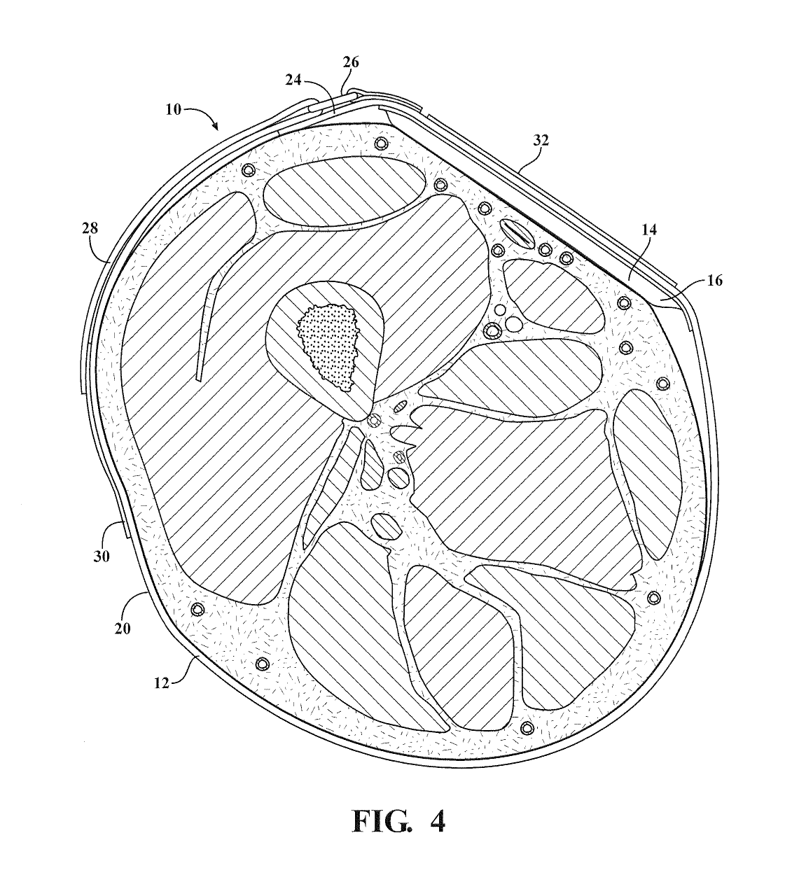

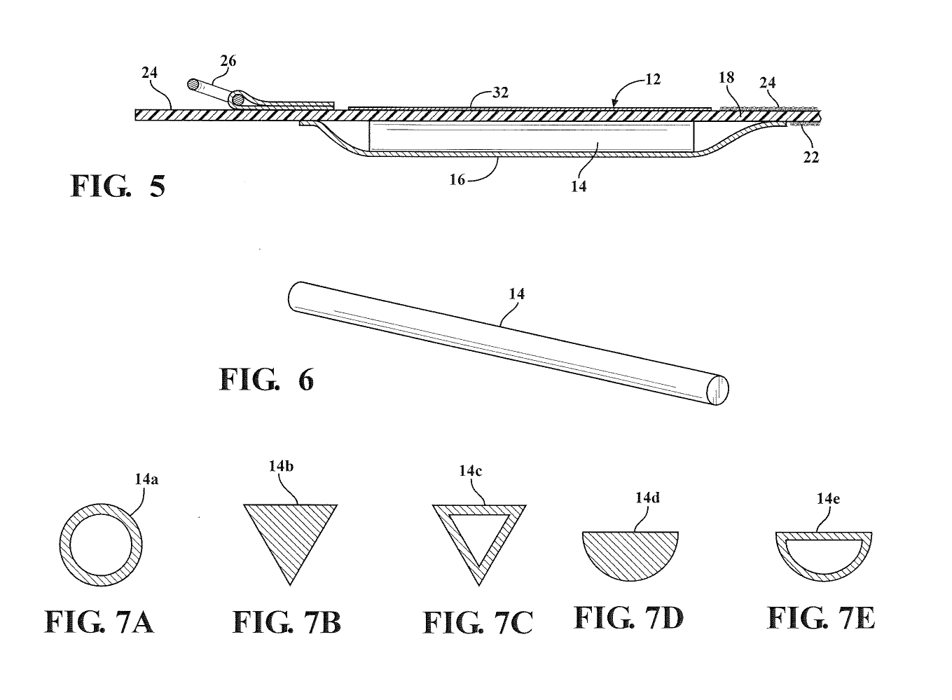

As shown best in FIGS. 2 and 4-6, the stay portion 14 may be received in a pocket 16 of the strap 12. The pocket 16 may be integral with the strap (e.g., a pocket formed in the strap per se) or the pocket 16 may comprise a patch of material that is sewn, glued or otherwise secured permanently or releasably (such as by hook and loop) to the strap 12 and made of the same or different material as that of the strap 12. The pocket 16 extends in the lengthwise direction of the strap 12 over a limited region and may completely envelop the stay 14 on all sides such that the stay 14 is no accessible from outside the pocket 16, or the pocket 16 may be formed with an optional opening or slit that enables the stay to be inserted into and removed from the pocket 16. The stay region of the device 10 defines a “high pressure” region

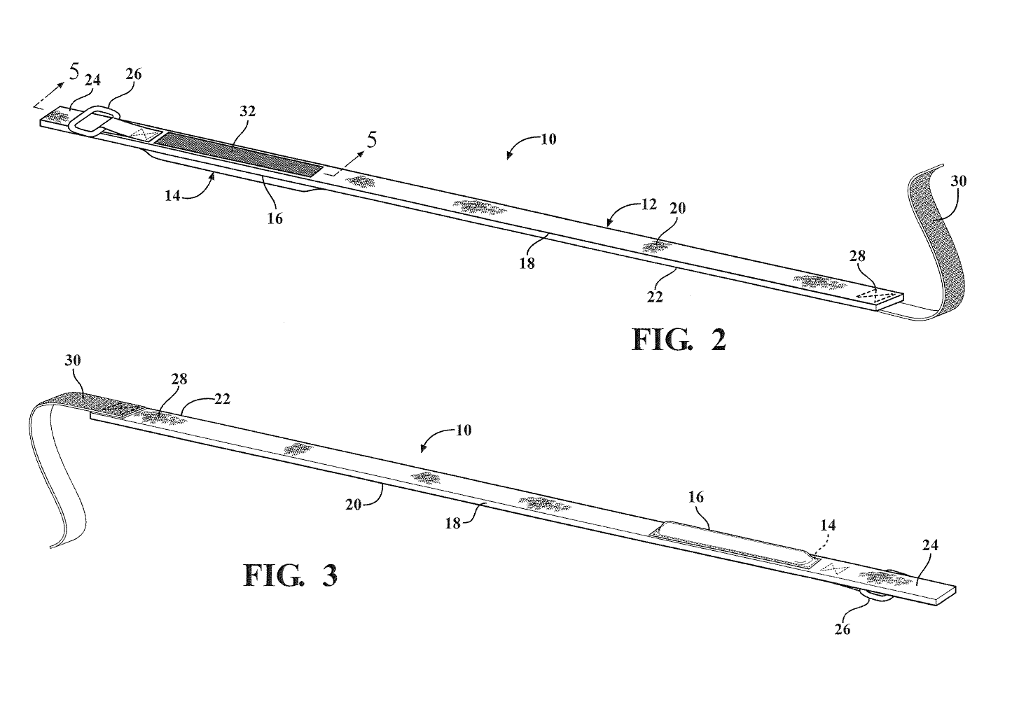

The strap 12 is made from a flexible and stretchy material. The material serves to limit the compression or cuff force that the strap 12 can exert on the limb of a user to below 200 mm/Hg. The material of the strap may be neoprene and tests have shown this to be effective to limit the cuff force to below 200 mm/Hg. The width of the strap may be in the range of 1 inch to 3 inches. The neoprene is very soft, flexible and stretchable. The neoprene foam core layer may have outer fabric layers secured to its outer surface as part of the strap construction. The outer fabric layers are likewise soft, flexible and stretchable with the neoprene. At least one of such outer layers may be loop pile material. Both sides of the strap may be covered in such loop pile material. FIGS. 2-5 show the strap 12 as having the neoprene core 18 and outer 20 and inner 22 loop pile layers covering both sides of the core 18.

The rigid stay 14 may be fabricated of a plastics material, such as nylon, glass-filled nylon, or other suitable material. The stay 14 may be fabricated of non-plastics materials such as wood, metal, ceramics or combinations thereof The stay 14 may be brittle or tough. The stay 14 may be entirely rigid and non-bendable or the stay 14 may be elastically or plastically bendable and shapeable as desired (but still relatively more rigid and less flexible than the strap portion which is unable to support its own weight in the lengthwise direction, whereas the stay is sufficiently stiff that it can).

The size of the stay 14 is also important but is not meant to be limiting to the scope of the invention. The stay 14 has a width that is considerably narrower that its length. The reduced width has the effect of concentrating the applied forces in a smaller area relative to the strap 12 and thus increasing the cuff pressure exerted on the limb in the area of contact immediately under the stay 14 relative to that applied by the strap 12. With increased rigidity and decreased surface area relative to the strap 12, the stay 14 amplifies the effective cuff pressure where it makes contact with the limb.

The outer cross-sectional shape of the stay 14 may be round (i.e., cylindrical in overall lengthwise outer shape). This is illustrated in FIG. 6. The outer diameter of the stay 14 may range from ⅛ inch to 1 inch, and may for example be ¼ inch in outer diameter. The cross-sectional shape does not have to be truly round, but may be triangular (FIG. 7B) with sharp or rounded edges, D-shaped (FIG. 7D) or other geometric shape that applies a desired linear concentrated contact force to the limb in its site of contact. The stay may be solid in cross section or tubular (FIGS. 7A, 7c and 7E).

The stay 14 may be positioned adjacent a first end 24 of the strap 12 on the inner surface 22. A strap buckle 26 may be mounted on the strap 12 adjacent the first end 24. The strap buckle 26 may take on various forms (i.e., different types of buckle devices may be employed) and is provided for the purpose of receiving a free end of the strap 12 so the device 10 can be looped about the user's limb and for engaging and gripping the strap 12 as it is guided through the buckle 12 by application of a pulling force on the free end of the strap so as to act as a fulcrum point and also to support the strap 12 from slipping freely back through the buckle 26 if the tension applied to the strap by the user is reduced so as to enable one-handed tightening and operation of the device if necessary. The particular type of buckle selected may offer more or less resistance to reverse slippage of the strap during tightening, with all types being contemplated herein.

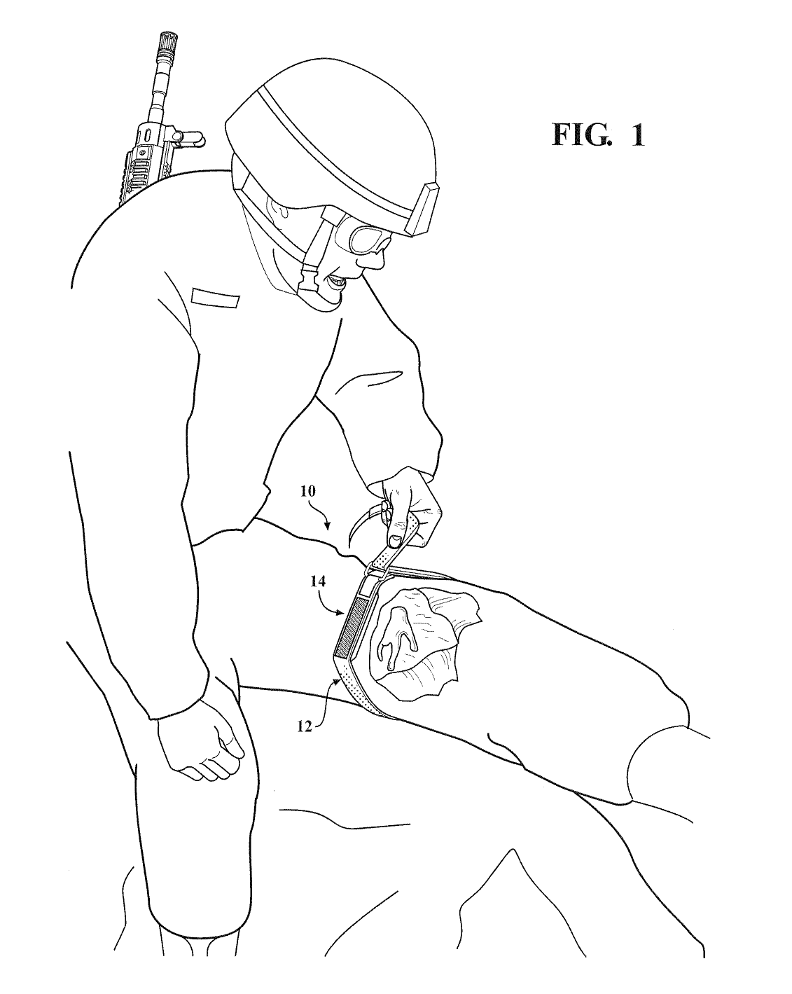

The type of buckle 26 according to the first embodiment is in the form of a D-ring. The D-ring 26 may be rounded or square (rectangular) and may be secured to the first end 24 of the strap 12 preferably a short distance inward from the end, as illustrated in FIGS. 1-5. The opposite end 28 of the strap 12 may be extended through the D-ring 26 in a tightening direction so as to form a closed circle with the strap, and then further drawn through the ring 26 and back on itself to tighten or cinch the strap 12 to reduce its effective diameter (FIGS. 1 and 4). When so wrapped about a limb (arm or leg), the stay 14 can be positioned to extend across a damaged arterial region or other severe bleed region of the limb where there is severe bleeding to be blocked (e.g., an artery that is partially or fully severed and which is bleeding internally or externally), at a location at or above the bleed site further toward the trunk of the body. See FIG. 1 as an example.

Once the stay 14 is positioned, the strap 12 can be tightly cinched by drawing the free end of the strap 12 back on itself through the ring 26. The strap 12 can be secured in the fully tightened position by means of hook and loop fasteners. The loop material may comprise the outer loop pile layer 20 of the strap 12, and this gives the strap great range of adjustability to fit limbs of various diameters (arms to legs, small to large in diameter).

As shown best in FIGS. 1-4, the securement feature of the strap can be provided by a length of hook pile tape 30. The hook pile tape 30 is flexible, but not stretchable as compared to the neoprene strap 12 material. The hook pile tape 30 may be sewn or otherwise fastened to the opposite end 28 of the strap 12 and preferably to the inside 22 surface, such that when fed through the ring 26, there is a smooth transition from the hook tape 30 to the neoprene strap 12, which strap is considerably thicker and whose raw edge may otherwise snag on the ring if the hook tape were secured to the outside when drawn through the ring 26. The hook tape 30 may be from 2 to 12 inches in length and the same or narrower than the strap 12. The hook tape 30 may have a slightly enlarged free end (e.g., a tab portion) which may be wider than the width of the ring 26, but flexible so as to slightly resist being pulled backward through the ring 26 once it is looped through, but which can be overcome and pulled free of the ring with effort.

When drawn tight, the strap 12 stretches and fits snug about the limb and presses the rigid stay 14 with concentrated greater force against the limb. The strap 12 is secured by drawing the hook tape 30 into engagement with the outer loop pile 20 of the strap 12 to securely but releasable hold the tourniquet in place on the limb.

The hook tape 30 is preferably a light color, such as tan or white, and the non-hook smooth side ends up facing outward once the tourniquet is applied. This provides a convenient writing surface for an attendant or the injured person to write information on the tape 30, such as the time the tourniquet was placed on the limb.

The neoprene strap 12 may be another color or graphic, such as black or camouflage. Since the stay 14 is on the inside surface of the strap 12, it is beneficial to include some form of marker on the outer surface to note where the stay is located for assisting in proper placement of the stay 14 on the injured region of the limb. For example, a patch of colored fabric or paint (e.g., red in color) may be applied to the outer surface 20 of the strap 12 to signify or associate the user with “wound” placement. FIGS. 1. 2, 4 and 5 illustrate such a wound or stay marker 32, and it may comprise, for example reflective red tape or fabric or paint. Other notification symbols, words and or colors may serve as indicia to help associate the orientation and proper placement of the stay region on the injured region of the limb.

The stretchy flexible strap material 14 is self-limiting in the amount of compression force (cuff force) that it can apply to the limb. It is less than 200 mm/Hg cuff pressure, and in practice applies no more than 150 mm/Hg cuff pressure. This same pressure is exerted on the stay 14 which is carried on and wrapped beneath the strap 12 against the arterial region. The rigid nature of the stay 14 coupled by its relatively and significantly reduced contact surface area (effectively line contact or bar contact with the limb) has the effect of amplifying the cuff pressure exerted by the stay 14 to above 300 mm/Hg, which is sufficient to stop arterial blood flow. In practice, the rigid stay 14 has been shown to exert up to 600 mm/Hg cuff pressure while the strap 12 away from the stay 14 exerted only about 150 mm/Hg. FIGS. 2 and 4 shoes the tourniquet in place on an injured leg, and it can be seen that a bleeding artery B is being compressed and pinched closed beneath the rigid stay portion 14 of the tourniquet that is applying 300 mm/Hg or more cuff pressure, while other undamaged collateral arteries C and/or veins V remain open beneath the flexible and stretchable strap 12 that is applying 200 mm/Hg or less cuff pressure.

The tourniquet 10 thus is constructed and acts to apply a variable or dual cuff pressure to the limb of a person in order to apply a cuff pressure of greater than 300 mm/Hg at the location of the rigid stay 14 in order to be effective to stop arterial bleed, while applying a cuff pressure of less than 200 mm/Hg of the strap 12 away from the stay 14 in order to maintain venous blood flow of the limb during the time the arterial flow is being blocked by the tourniquet.

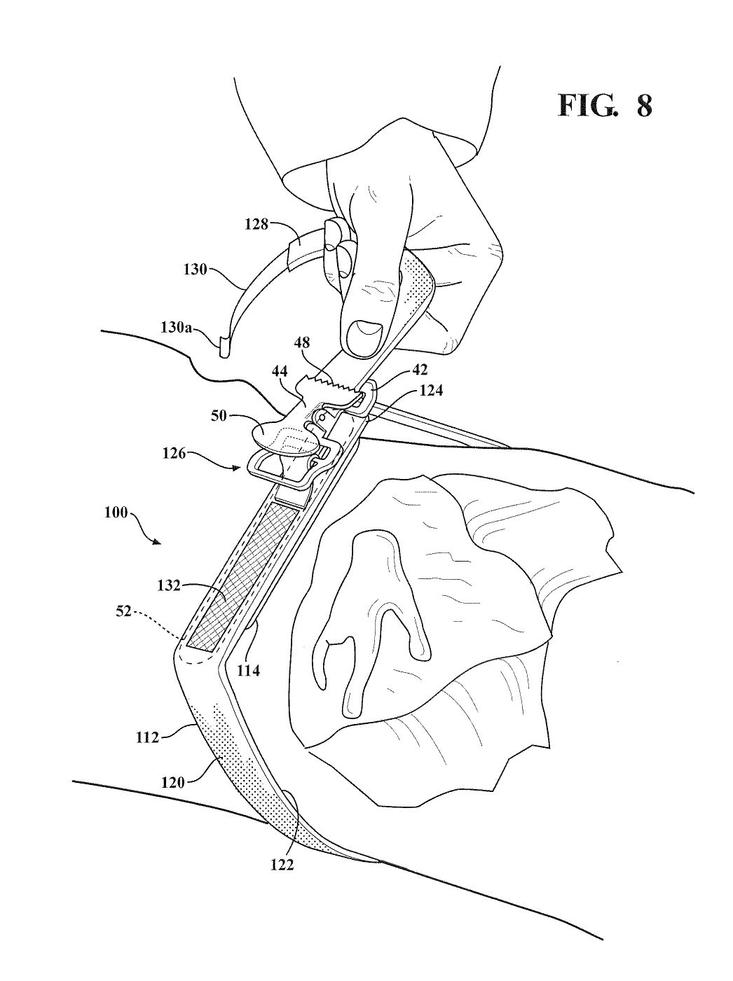

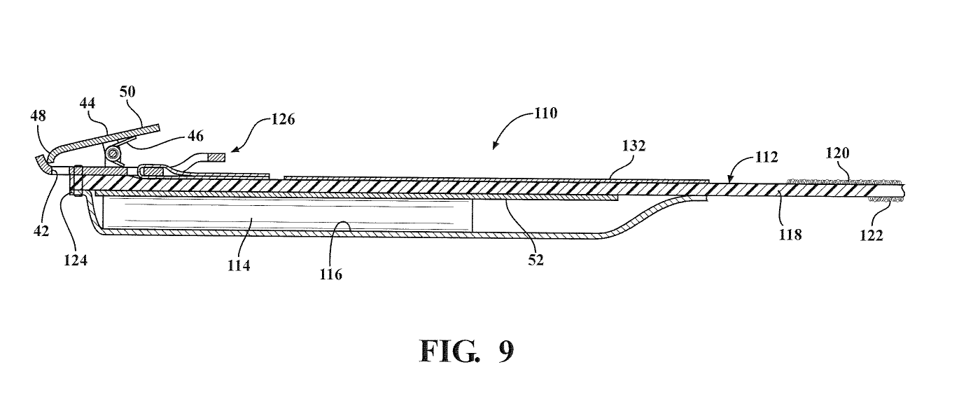

FIGS. 8 and 9 illustrate an alternative embodiment of the tourniquet device, wherein the same reference numeral are used to identify like features to that of the first embodiment, but are offset by 100. The tourniquet 110 is essentially the same as that of the tourniquet 10 of the first embodiment, except that the D-ring style buckle 26 has been replaced with an alligator spring clip buckle type device 126. In addition, a supplemental reinforcement member has been added to the pocket 116 as a supplemental backing to the stay member 114 within the pocket 116.

The spring clip buckle 126 is anchored in position on the outer surface of the strap 112 adjacent the one end 124. The spring clip buckle 126 may be secured by sewing, gluing, rivets, or other suitable means. The spring clip 126 includes a stationary ring portion 42 at a forward end thereof and a movable jaw 44 that is hinged at a location spaced from the ring portion 42 and constantly biased by a spring 46 to pivot the jaw 44 downwardly with constant spring force toward a closed position in cooperation with the ring portion 42. The jaw 44 has a forward serrated edge 48 that faced the ring portion 42 and the position of the forward edge 48 may be adjusted by applying an opening force to a release lever portion 50 of the jaw 44 projecting in the opposite direction of the edge on the opposite side of the pivot axis that is sufficient to overcome the closing force exerted by the spring 46. Pressing on the lever portion lifts the forward edge 48 away from the ring portion 42 and has the effect of increasing or decreasing the gap or throat defined by the space between the free edge 48 and ring portion 42. When in an unassisted rest state, the spring 46 may force the free edge 48 into engagement with the ring portion 42 such that the throat is biased toward a fully closed condition, but can be selectively opened by bodily pressing on the release lever 50. Alternatively, the rest state of the buckle 126 may be configured such that there is a small gap between the free edge 48 and ring portion 42 so that the throat remains at least slightly opened.

The free end of the strap 112 is fed through the throat of the ring portion 42 from below. This may require that the user press on the release lever 50 to overcome the spring force and open the throat sufficiently to accept passage of the strap. As illustrated in FIG. 8, the initial lead end of the strap will actually be the free end of the hook tape 130, and this free end may advantageously be curled at 130a as illustrated in a direction that, when inserted into the throat from below, curls and guides the hook tape 130 outwardly and away from the underside of the jaw 44. The curl 130a may be imparted to the tape 130 by heat setting and serves to self-bias the end of the tape to a J-shape, but which also enables the curled end to lie flat once the hook tape 130 is eventually secured. The curl 130a makes it easier to thread the free end without obstruction from the jaw 44.

Once the strap 112 is started, the lever 50 may be released allowing the free edge 48 of the jaw 44 to pivot downwardly and pinch the strap 112 between the free edge 48 and the ring portion 42. Because of the orientation and pivot angle of the jaw 44 (the jaw 44 extends toward and downward relative to the ring portion 42 and strap 112), a bodily tension force applied to the free end of the strap as in FIG. 8 in the feed direction has the effect of applying a lifting force on the jaw 44 to open the throat sufficiently to enable the strap to continue to slip through the throat of the buckle 126. However, once the applied tension force is released, the constant closing force exerted by the spring 46 pivot the free end 48 downward to tightly clamp the strap 112. A further beneficial feature to this type of buckle is that orientation of the jaw 44 and free edge 48 prevent the strap 112 from slipping back through the buckle 126 in the reverse direction of feed. This one-way locking feature enables the user to tighten the strap 112 with one hand, as illustrated in FIG. 8, and permits the user to release the strap 112, either intentionally or inadvertently, without concern for the strap 112 loosening or slipping back through the buckle 126 in the opposite direction. If the strap 112 needs to be repositioned or otherwise loosened for any reason, the user may simply press on the release lever 50 to open the jaw 44 and release the gripping force on the strap 112.

Turning now to the other notable feature of the second embodiment of the tourniquet device 110, the pocket 116 contains the stay 114 as in the first embodiment and it projects inwardly from the inside surface of the strap 112, but also includes a supplemental rigid backing strip 52. The backing strip 52 is arranged in the pocket 116 in between the stay 114 and the strap 112. The backing strip 52 is fabricated of a rigid or semi-rigid material and has a generally flat, planer profile. The backing strip 52 may be flexible and is wider than the stay 114. The width of the backing strip 52 may be slightly narrower than the strap 112. The backing strip 52 has a length that is equal to or greater than that of the stay 114. The backing strip may extend beyond the rearward end (i.e., away from the one end 124 of the strap 112) by several inches, or about ⅓ of the length of the stay 114. The backing strip 52 helps concentrate the force of the strap 112 on the stay 114 and also helps stabilize the position of the stay 114. The backing strip 52 and stay 114 may be formed as one piece if desired. The buckle 126 may also be united directly to the backing strip 52 either as one piece or via mounting rivets or the like.

In both embodiments, the stay 14, 114 presents a generally linear and relatively smaller contact area compared to that of the strap 12, 112. The relative rigidity and smaller contact area of the stay 14, 114 contribute to its relatively higher cuff pressure compared to that able to be produced by the wider, flexible and stretchable strap 12, 112. In other words, the structure of the tourniquet 10, 110 (selection of materials and arrangement of parts) is directly responsible for the resultant performance of the tourniquet 10, 110 when applied to the limb. This has the advantage of minimizing the level of training needed to use the device and also minimizes the chances of misusing the device under the stressful conditions of an emergency. The simple construction also lends to a certain intuitive understanding as to how the device should work, even without prior training. The simplicity of the design, the provision of the indicia and usage of common buckle devices, hook and loop fastening systems, etc. aid in the ready understand of the function and operation of the device.

The foregoing description is exemplary rather than limiting in nature. Variations and modifications to the disclosed embodiment may become apparent to those skilled in the art are herein incorporated within the scope of the invention. The invention is defined by the appended claims.

What is claimed is:

1. A tourniquet, compromising: a strap of flexible stretchable material having a buckle disposed adjacent one end and having an opposite free end of said strap extendible through said buckle to enable said strap to be wrapped about a person's limb in proximity to a bleeding wound in need of a tourniquet; a rigid stay portion disposed on an inner side of said strap and extending over only a portion of a length of said strap and acting to exert a greater cuff pressure to the person's limb in relation the a lesser cuff pressure exerted by said strap away from the region of said stay portion.

2. The tourniquet of claim 1, wherein said rigid stay portion projects inwardly of said strap in relation to said inner surface of said strap.

3. The tourniquet of claim 1 wherein said strap is fabricated at least in part of neoprene material.

4. The tourniquet of claim 3 wherein said strap includes mateable hook and loop closure portions.

5. The tourniquet of claim 4 wherein said loop portion is provided on an outer surface of said neoprene strap and said hook portion is provided by a strip of hook tape extending from an end of said neoprene strap material.

6. The tourniquet of claim 5 wherein said hook tape is white in color and smooth on a non-hook side thereof.

7. The tourniquet of claim 4 wherein said strap includes indicia on an outer surface of said strap opposite said stay to indicate the location of said stay along said strap.

8. The tourniquet of claim 1 wherein said buckle comprises a spring clip having a jaw spring-biased toward a closed position and permitting said strap to be slid through said buckle in a strap tightening direction and restraining said strap from being slid in the opposite direction with greater force without manual release of said jaw.

9. The tourniquet of claim 1 wherein said stay is disposed in a pocket.

10. The tourniquet of claim 9 including a rigid backing strip disposed in said pocket between said strap and said stay.

11. The tourniquet of claim 10 wherein said backing strip is wider than said stay.

12. The tourniquet of claim 11 wherein said backing strip is longer than said stay.

13. The tourniquet of claim 1 wherein said stay presents a smaller contact surface area than that of said strap.

14. The tourniquet of claim 13 wherein said stay is disposed adjacent said one end of said strap.

15. The tourniquet of claim 14 wherein said buckle is disposed above said stay on the opposite side of said strap.

16. The tourniquet of claim 1 wherein the stay is generally cylindrical.

17. A tourniquet, comprising: a length of flexible and longitudinally stretchable neoprene strap material extending longitudinally from one end to an opposite end; a length of flexible and relatively non-stretchable hook tape material extending from said opposite end of said neoprene strap material to a free end; a spring clip buckle disposed on an outer surface of said neoprene strap material adjacent said one end of said neoprene strap material and including a pivoting jaw extending toward said free end and spring-biased toward a closed position relative to a ring portion of said spring clip; a relatively rigid stay portion disposed on an inner side surface of said neoprene strap material adjacent said one end; said stay extending along only a portion of the length of said neoprene; said stay being disposed in a pocket and presenting a generally linear, relatively rigid contact surface that projects inwardly of said inner surface of said neoprene strap material; said outer surface of said neoprene strap material being provided with a loop pile finish that is functionally mateable with the hook tape material to selectively and releasably secure the hook and loop materials together; and wherein said stay acts to exert a first relatively high cuff pressure when the tourniquet is wrapped about a user's injured limb sufficient to stop blood flow from a severely injured region of the limb, and the neoprene strap material acts to exert a second lower cuff pressure that is sufficiently low to permit venous blood circulation across the strap in the adjacent uninjured regions of the limb.

18. A tourniquet, comprising a flexible, stretchable strap wrappable about a person's severely injured limb and a relatively rigid, relatively non-stretchable rigid member disposed on a portion of the strap, wherein said relatively rigid member is operative to apply a first relatively high cuff pressure to the limb sufficient to stop blood flow from the severely injured region of the limb, and wherein said flexible and stretchable strap is operative to exert a relatively lower cuff pressure than that of the rigid member to the portion of the limb surrounding the injured region to permit venous blood flow across said strap.

19. A method of applying a tourniquet to a limb of a person suffering blood loss from a severe wound, comprising; wrapping a flexible, stretchable strap portion of the tourniquet about the user's limb adjacent the wound; positioning a relatively rigid stay portion of the tourniquet immediately above or at the wound site where blood loss is occurring; tightening the strap until sufficient cuff pressure is applied by the stay portion to stop the blood loss and wherein the flexible stretchable strap exerts a relatively lower cuff pressure to the surrounding region of the limb sufficient to permit venous blood flow across the strap in the uninjured region of the limb.

20. The method of claim 1, wherein the wrapping and tightening of the strap is performed by looping the strap through a spring clip buckle on the strap and drawing the loop tight about the limb by pulling on the strap with one hand to draw the strap through the buckle.

21. The method of claim 20 including securing a pulled through portion of the strap with hook and loop closure portions of the tourniquet.
